(19) United States
(12) Patent Application Publication
(10) Pub. No.: US 2010/0179481 A1
(43) Pub. Date: Jul. 15, 2010

(54) SECUREMENT DEVICE FOR A NEONATE

(75) Inventors: Steven Bierman, Del Mar, CA (US); Richard Pluth, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Venetec International, Inc., Covington, GA (US)

(21) Appl. No.: 12/676,340

(22) PCT Filed: Sep. 10, 2007
(86) PCT No.: PCT/US2007/078058

§ 371 Date: Mar. 3, 2010

(51) Int. Cl. A61M 25/02

(52) U.S. Cl. 604/177; 604/180

(57) ABSTRACT

A securement device (10) for securing a medical line to a neonate includes first and second anchors (20a), (20b) disposed in opposing relationship to each other. Each anchor (20) comprises an anchor pad (22), a retainer (26), and a connector (24) connecting the anchor pad (22) and retainer (26). The connectors (24) extend in a direction substantially normal to the anchor pads (22) and grip the medical line from opposing directions. The device (10) is particularly adapted for use in connection with neonates and provides a small securement surface to fit onto the small body locations of a neonate. The device (10) is also particularly adapted for applications in which the medical line enters the body in a direction generally normal to the surface of the body, such as umbilical, nasogastric, or endotracheal applications.

### BACKGROUND OF THE INVENTION

1. Field of the Invention

This application relates to a medical line securement device. More particularly, this application relates to a device that stabilizes an upright elongated medical line against a neonate's skin.

2. Description of the Related Art

Premature babies usually require assistance to breathe and eat, and thus, are often fully instrumented. An endotracheal tube typically extends down the trachea of the neonate to supply oxygen to the lungs from a ventilation machine. These babies also are usually fed intravenously by an intravenous (IV) catheter inserted into a vein, usually on the dorsal side of the neonate's hand or forearm. In some cases, a naso-gastric tube may be inserted through the neonate's nose and used either for feeding/hydrating purposes or to drain gastric secretions. An arterial catheter may also be inserted on the ventral side of the neonate's hand for purposes of monitoring blood oxygenation.

The umbilical stump additionally is often catheterized. An umbilical catheter is inserted through the umbilical stump and is placed into the neonate's circulation system, with the assistance of fluoroscope or x-ray visualization, to precisely position the distal end of the catheter. The umbilical catheter allows medication to be directly introduced into the bloodstream and permits routine blood sampling, without having to frequently puncture the delicate skin of the neonate.

Each of these catheters and tubes is precisely positioned, and the position of each is fixed relative to the neonate to prevent migration or dislodgment. Healthcare personnel usually secure the IV, arterial and/or umbilical catheters using tape, tape bridges, and sometimes sutures. Endotracheal and/or naso-gastric tubes are also affixed with tape. Tape application, especially the construction of tape bridges, is a time-consuming and therefore expensive process. Furthermore, the catheters and tubes remain in place for several days or weeks, and often require repositioning and/or replacement on a periodic basis. The tape thus must be removed and reapplied to the neonate's delicate skin.

Tape often collects dirt and other contaminants. Normal protocol therefore also requires periodic tape changes to inhibit bacteria and germ growth at the securement site. Frequent tape changes exfoliate the upper layers of the neonate's skin. Such exfoliation of the upper layers of skin can lead to abrasions on the neonate's skin because the skin is so thin, sensitive and fragile. Moreover, infection and disease can occur as a result of such skin wounds owing to the relatively undeveloped nature of a neonate's immune system.

### SUMMARY OF THE INVENTION

The systems and methods of the present invention have several features, no single one of which are solely responsible for its desirable attributes. Without limiting the scope of this invention as expressed by the claims which follow, its more prominent features will now be discussed briefly. After considering this discussion, and particularly after reading the section entitled “Detailed Description of the Preferred Embodiments,” one will understand how the features described herein provide advantages over traditional medical line securement systems.

An aspect is a securement device for securing a medical line having a longitudinal axis in a generally upright manner near an insertion site. The device includes at least two anchors. Each anchor has an anchor pad, a retainer, and a flexible connector extending between the anchor pad and the retainer. The retainer of the first anchor is configured to grip the medical line at a first location along the longitudinal axis. The retainer of the second anchor is configured to grip the medical line at a second location, different than the first location, along the longitudinal axis. The flexible connectors are radially offset from each other about the longitudinal axis of the medical line at least when the medical line is secured to the patient.

Another aspect is a method of securing a medical line to a patient near an insertion site. The method comprises securing a first anchor to a patient. The first anchor has a first anchor pad, a first retainer, and a first flexible connector extending between the first anchor pad and the first retainer. The first retainer is configured to grip the medical line at a first location along the longitudinal axis of the medical line. The method further comprises securing a second anchor to a patient at a different angle with respect to the insertion site than the first anchor. The second anchor has a second anchor pad, a second retainer, and a second flexible connector extending between the second anchor pad and the second retainer. The second retainer is configured to grip the medical line at a second location along the longitudinal axis of the medical line.

Another aspect is a securement device for securing a medical line that has a longitudinal axis in a generally upright manner near an insertion site. The device comprises an anchor pad and a retainer configured to adhere to the medical line along a longitudinal axis. The device further comprises a flexible connector between the anchor pad and the retainer and a first adhesive and a second adhesive.

Another aspect is a securement device for securing a medical line that has a longitudinal axis in a generally upright manner near an insertion site. The device comprises an anchor pad that has an adhesive on at least a portion of a lower surface and a retainer configured to adhere to the medical line along a longitudinal axis. The device further comprises a flexible connector between the anchor pad and the retainer. The flexible connector has at least one receptacle through which at least a portion of the adhesive directly adheres the anchor pad to the patient.

### BRIEF DESCRIPTION OF THE DRAWINGS

The above-mentioned and other features of the invention will now be described with reference to the drawings of an embodiment which is intended to illustrate but not to limit the invention. The drawings contain the following figures:

FIG. 1 is a perspective view of a medical line securement device configured in accordance with a preferred embodiment of the present invention, and illustrating an exemplary use of the device attached to a neonate's abdomen and securing an umbilical catheter.

FIG. 2 is an enlarged perspective view of the securement device of FIG. 1 showing the portion of FIG. 1 indicated by line 2-2.

FIG. 3 is a side elevation of the securement device of FIG. 1 shown securing the umbilical catheter.

FIG. 4 is a perspective view of the securement device of FIG. 1.

FIG. 5 is a top plan view of the securement device of FIG. 1.

FIG. 6 is a cross-sectional view of the securement device of FIG. 5 taken along line 6-6.

FIG. 7 is a cross-sectional view of the securement device of FIG. 5 taken along line 7-7.

### DETAILED DESCRIPTION OF THE PREFERRED EMBODIMENTS

The illustrated embodiment of the medical line securement device 10 is disclosed in the context of use with an exemplary medical infusion line 12 comprising an umbilical venous catheter (UVC). The principles of the present invention, however, are not limited to UVC lines. Instead, it will be understood by one of skill in the art, in view of the present disclosure, that the securement device disclosed herein can also be used with other types of medical lines, such as with other catheters, fluid drainage and delivery tubes, electrical leads and the like. It is also understood that the skilled artisan can readily adapt the securement device for use in other applications to retain many different types of medical lines to a patient. Thus, the illustration and description of the medical line securement device in connection with the UVC line 12 is merely exemplary of one possible application of the securement device. The present invention also may be used at other locations on the neonate's body. For instance, the securement device may be used on the neonate's face to secure an endotracheal tube or a naso-gastric tube.

The securement device 10 has particular applicability in connection with securing a medical line to a neonate. The device 10 can be configured to provide a small securement surface to fit onto the small body of the neonate. The device, however, can also be successfully utilized with any medical patient, notwithstanding the size or age of the patient, although it may be preferable to increase the size of the device and the resulting contact area with the patient. Thus, the illustration and description of the securement device in context of use with a neonate is merely exemplary of one possible application of the securement device.

The medical line securement device 10 provides for secure attachment of the medical line 12 to the neonate's skin without the use of surgical tape, thereby limiting the risk of irritating or infecting the neonate's skin. The securement device 10 may include one or more types of adhesives. The device 10 further provides stabilization of the medical line 12 for applications in which the medical line enters the body in a direction generally normal to the surface of the body, such as umbilical, nasogastric, or endotracheal applications.

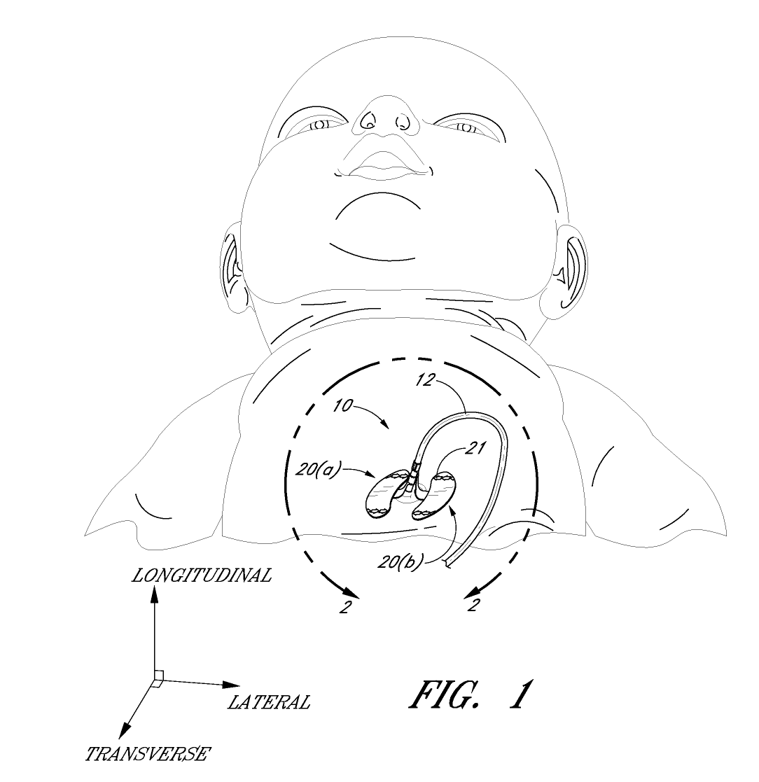

To assist in the description of these components of the securement device 10, the following coordinate terms are used. A “longitudinal axis” is generally parallel to the section of the medical line secured by the securement device 10. For applications in which the medical line enters the body in a direction generally normal to the surface of the body, then, the longitudinal axis will be generally normal to the surface of the body. A “lateral axis” is normal to the longitudinal axis and is generally parallel to the plane of the minor axes of anchor pads 22(a), 22(b) illustrated in FIG. 1. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. A detailed description of the securement device 10 and associated methods of use now follow.

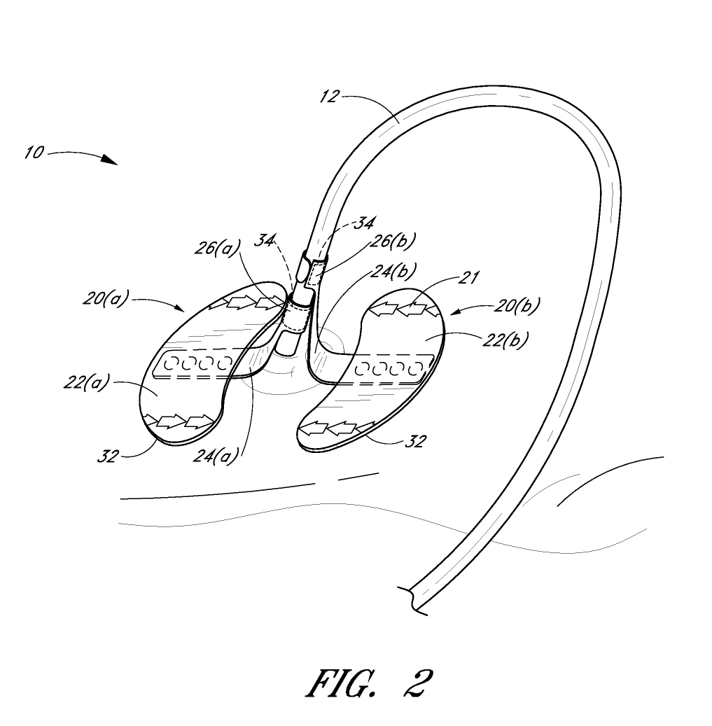

With reference to FIGS. 1 and 2, the medical line securement device 10 is shown applied to the skin of a neonate and securing a medical line 12. The device 10 principally comprises anchors 20(a) and 20(b). In use, the anchors 20(a), 20(b) are disposed at different locations about a longitudinal axis of the medical line 12 in order to grip the medical line 12 from different sides. In the illustrated embodiment, for example, the anchors 20(a), 20(b) are disposed in an opposing relationship to each other about the longitudinal axis of the medical line 12.

As best seen in FIG. 2, the anchor 20(a) generally includes anchor pad 22(a), connector 24(a), and retainer 26(a). Similarly, the anchor 20(b) includes anchor pad 22(b), connector 24(b), and retainer 26(b). Each of the anchor pads 22(a), 22(b) may include one or more types of adhesives for adhering the anchor pad to the neonate's skin.

Each of the anchor pads 22(a), 22(b) may comprise one or more types of adhesive 32 on all or a portion of the bottom surface of each anchor pad 22(a), 22(b). Employing a less costly adhesive in one or more regions of the anchor pad 22(a), 22(b) while employing a more costly adhesive only where needed is advantageous with respect to the entire cost of the securement device 10.

The adhesive(s) 32 may be provided across the entire bottom surfaces of the anchor pads 22(a), 22(b), or may be provided only in certain regions, such as the outer perimeters of the anchor pads 22(a), 22(b) or a central region of the anchor pads 22(a), 22(b). Additionally or in the alternative, the adhesive(s) 32 may be provided in a discontinuous pattern, such as a striped or dotted pattern, on the undersides of the anchor pads 22(a), 22(b).

Each anchor pad 22(a), 22(b) may include one or more types of adhesives. The adhesives may have different material properties, including for example adhesive strength or peal strength. For extremely secure adhesion to the patient, the adhesive(s) 32 provided on the undersides of the anchor pads 22(a), 22(b) may comprise a very strong adhesive, such as a VHB™ adhesive provided by 3M Corporation. Each anchor pad 22(a), 22(b) may include a first adhesive in a first region of the anchor pad and a second adhesive in a second region of the same anchor pad. The first and second regions may lie adjacent to each other or partially overlap.

For embodiments having overlapping adhesives, the first adhesive may cover the entire bottom surface of the anchor pad 22(a), 22(b) with the second adhesive covering only a portion of the bottom surface of the first adhesive. This may be advantageous for manufacturing purposes since it ensures that the entire bottom surface is covered with adhesive. Alternatively, the first adhesive may cover a first region of the anchor pad, such as the outer perimeter, with the second adhesive being disposed so as to be adjacent to the first region and cover the central region of the anchor pad. With adjacent as opposed to overlapping adhesives, the manufacturing process may require additional masking to ensure that the adhesives do not overlap.

Having multiple types of adhesives on a single anchor pad 22(a), 22(b) may be advantageous for handling purposes or to provide higher peal strength between the patient's skin and the securement device 10 in a select region of the anchor pad 22(a), 22(b). For example, a high strength adhesive may be disposed in a center region and a lower strength adhesive disposed outside of the center region. This arrangement may be advantageous for handling purposes since only the high strength adhesive is exposed in the center region making it less likely that the healthcare provider would come in contact with the high strength adhesive.

The locations of the different adhesives may also be selected to make a selected region of the anchor pad 22(a), 22(b) less likely to separate from the patient. For example, a higher-strength adhesive may be used around the perimeters of the anchor pads 22(a), 22(b) to prevent separation of the anchors 20(a), 20(b) from the skin of the patient around the edges of the anchor pads 22(a), 22(b). Such an arrangement may further prevent entry of contaminants underneath the anchor pads 22(a), 22(b) while a lower-strength adhesive may be used in the center regions.

The locations of the different adhesives may also be selected to further inhibit unwanted longitudinal movement of the medical line 12. For example, a higher-strength adhesive may be employed near the regions of the anchor pads 22(a), 22(b) that are adjacent to the medical line 12 to prevent separation of the anchors 20(a), 20(b) from the skin of the patient in the regions of the connectors 24(a), 24(b). The regions of the anchor pads 22(a), 22(b) which overlap with the connectors 24(a), 24(b) may be exposed to higher tensile loads and accordingly may employ an adhesive with a higher peal strength. In another embodiment, regions of the anchor pad 22(a), 22(b) near the connectors 24(a), 24(b) and outer perimeters of the anchor pads 22(a), 22(b) are covered with the higher-strength adhesive 32 while the remaining regions are covered with a low-strength adhesive.

The connectors 24(a), 24(b) extend from the anchor pads 22(a), 22(b) and connect the pads 22(a), 22(b) to the retainers 26(a), 26(b), respectively. The connectors 24(a), 24(b) are preferably flexible so as to allow the connector to follow the contours of the patient between the anchor pads 22(a), 22(b) and the retainers 26(a), 26(b). Of course the connectors 24(a), 24(b) need not be flexible and instead may have a rigid curvilinear shape. The rigid shape preferably follows the expected surface contour of the patient.

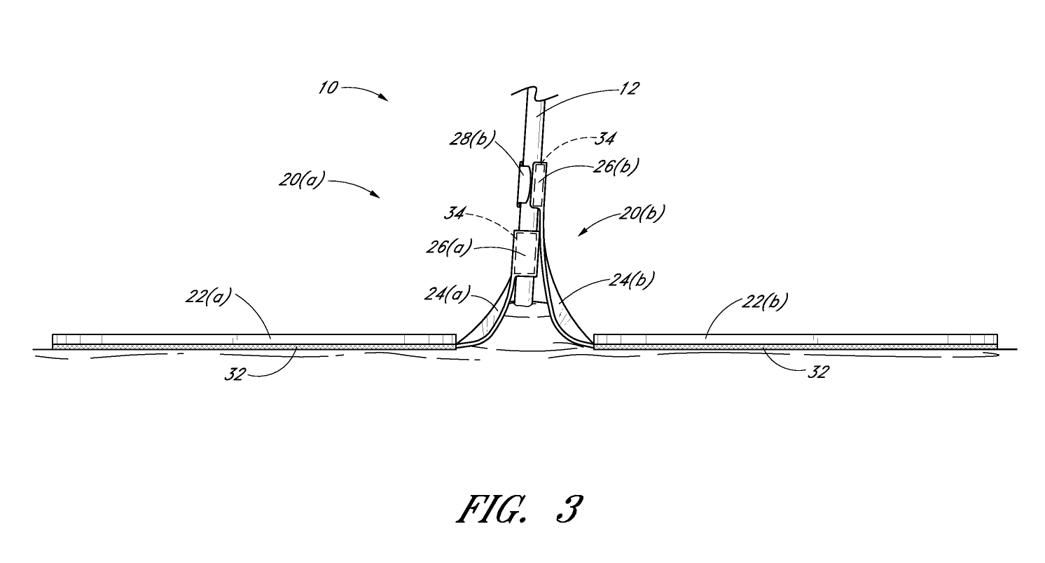

As best seen in FIG. 3, in use, the connectors 24(a), 24(b) may initially extend in a lateral direction from the anchor pads 22(a), 22(b) toward the indwelling medical line 12. The connectors 24(a), 24(b) may then be bent up in a generally longitudinal direction to allow the retainers 26(a), 26(b) to grip the medical line 12.

Preferably, at least portions of the anchor pads 22(a), 22(b) adhere to the patient's skin on opposite lateral sides of the medical line 12. In use, the longitudinal height of one of the anchors 20(a), 20(b) is preferably greater than the height of the other anchor 20(a), 20(b). This arrangement allows the retainers 26(a), 26(b) to grip the medical line 12 at different longitudinal locations along the medical line 12. In this way, the stability of the secured medical line 12 is enhanced relative to the anchor pads 22(a), 22(b). Of course the retainers 26(a), 26(b) may be disposed along the longitudinal axis so that portions of the retainers 26(a), 26(b) overlap on the medical line 12.

In the illustrated embodiment, the connector 24(b) is longer than the connector 24(b). The retainer 26(b) grips the medical line 12 at a location distal (with respect to the neonate's body) of the location where the retainer 26(a) grips the medical line 12. For example, as shown in the figure, the connector 24(b) is longer than the connector 24(a) by at least the length (measured in the longitudinal direction) of the retainer 26(a). The retainers 26(a), 26(b) are configured to retain the medical line 12 from different sides, such as from opposing sides of the medical line 12. With this configuration, the anchors 20(a), 20(b) cooperate to limit movement of the medical line 12 in the longitudinal direction as well as in the lateral and transverse directions.

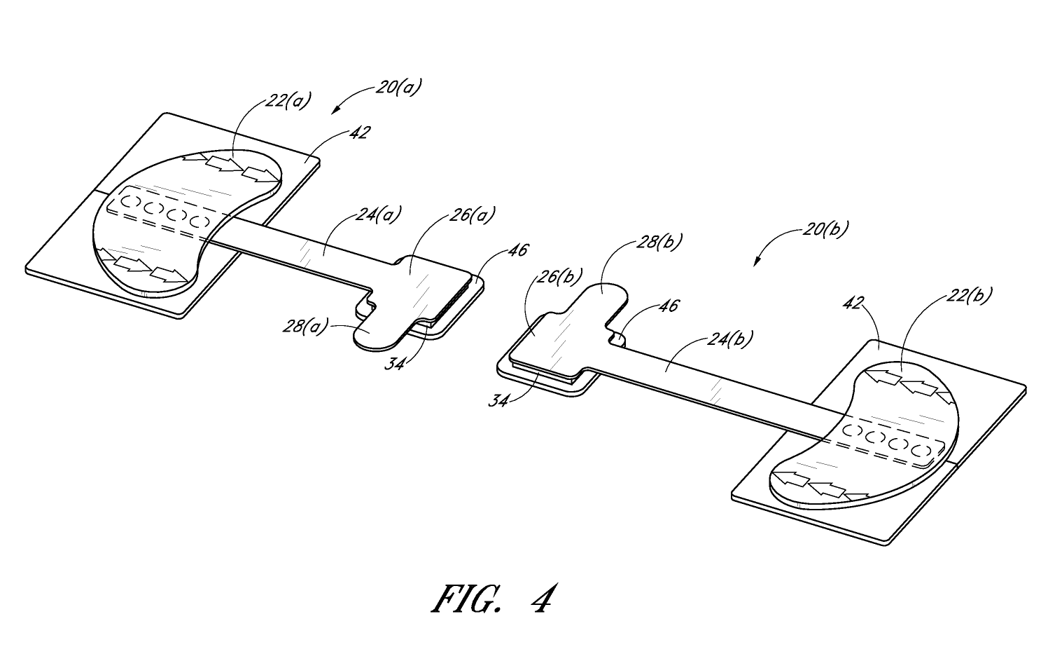

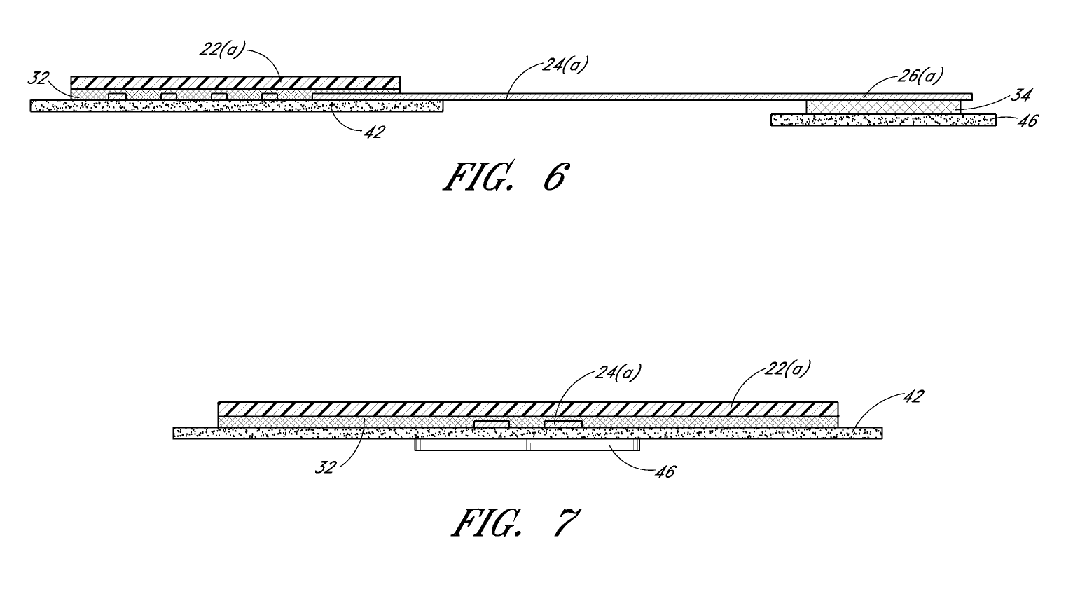

Referring now to FIGS. 4-7, the anchors 20(a), 20(b) are shown generally as supplied to the medical practitioner. As shown in FIG. 4, the anchors 20(a) and 20(b) may be supplied in an essentially flat configuration, with the connectors 24(a), 24(b) extending horizontally from the middle portion of the anchor pads 22(a), 22(b) to the retainers 26(a), 26(b), respectively.

The anchor pads 22(a), 22(b) may be provided with a removable liner 42. The liner 42 may comprise a polyester film liner or other material. The liner 42 is configured to protect the adhesive 32 prior to application of the anchor pads 22(a), 22(b) to the neonate's skin. The liners 42 may extend beyond the footprint of the anchor pads 22(a), 22(b), and/or may be provided with a slit down the middle to facilitate their removal.

The retainers 26(a), 26(b) may be provided with a removable liner 46 configured to protect the adhesive 34 prior to securement of the retainers 26(a), 26(b) to the medical line 12. The liners 46 may extend beyond the footprint of the retainers 26(a), 26(b), and/or may be provided with a slit down the middle to facilitate their removal.

Each of the anchor pads 22(a), 22(b) may comprise a flexible material configured to conform to the contours of the neonate's body at the attachment surface. The anchor pads 22(a), 22(b) may also be printed with arrows 21 or other suitable indicators to show the proper placement of the pads 22(a), 22(b) with respect to one another on the neonate. Although the illustrated embodiment shows two separate anchor pads 22(a), 22(b) having the same kidney-shaped configuration, the anchor pads may have any other shape or configuration consistent with their intended use. For example, although the illustrated anchor pads 22(a), 22(b) may be somewhat spaced apart when securing a medical article, the pads may instead be configured to touch or partially overlap each other when properly positioned on a patient. Alternatively or in addition, the anchor pad 22(a) may have a different shape than the anchor pad 22(b).

The anchor pads 22(a), 22(b) may be provided with one or more interlocking tabs and/or grooves configured to indicate proper placement with respect to one another on a patient's skin. Additionally, one or both of the pads 22(a), 22(b) may have a more rigid, perhaps curved configuration adapted for a particular location (such as a chin) on a patient's body. The anchor pads 22(a), 22(b) may also be connected to or continuous with one another, for example in a semicircular or horseshoe-shaped configuration about the longitudinal axis of the medical line 12.

The anchor pads 22(a), 22(b) may comprise, for example, a flexible synthetic fabric such as Hy-Tape® adhesive tape. The adhesive 32 on the underside of the anchor pads 22(a), 22(b) may comprise any adhesive suitable for medical use on human skin, such as a zinc oxide-based adhesive particularly adapted for use with delicate skin. Additional adhesive materials and configurations are disclosed in U.S. Pat. No. 6,866,652, entitled “Medical Line Securement Device For Use With Neonates,” at column 6, line 38, through column 7, line 23, which paragraphs as well as the entire disclosure are hereby incorporated by reference.

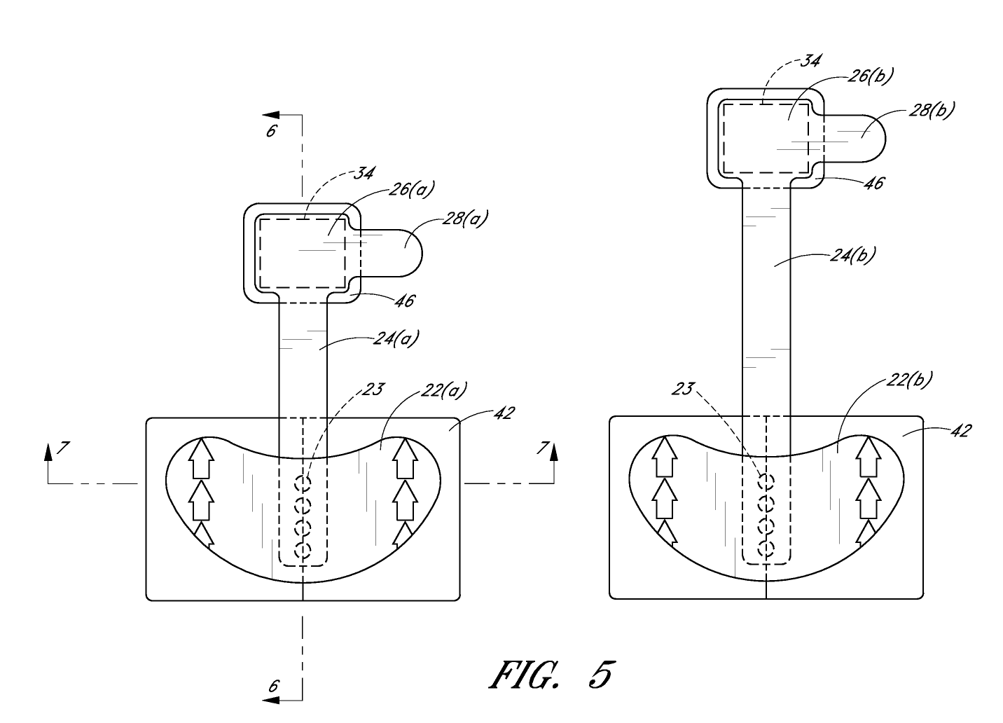

As best illustrated in FIG. 5-7, the connectors 24(a), 24(b) may comprise lengths of flexible or semi-flexible material, such as thin strips of a flexible polyester film like PET film. The connectors 24(a), 24(b) may extend from underneath the anchor pads 22(a), 22(b) (see FIG. 5) to the retainers 26(a), 26(b). The connectors 24(a), 24(b) may be attached to the anchor pads 22(a), 22(b) using an adhesive, such as the adhesive 32. In the region underneath the anchor pads 22(a), 22(b), the connectors 24(a), 24(b) may be provided with one or more holes 23 configured to allow the adhesive 32 to adhere to the neonate's skin through the connectors 24(a), 24(b). The holes 23 may comprise a series of substantially circular holes, as shown in the figures, or may comprise one or more slits disposed longitudinally, laterally, or at an angle with respect to the connectors 24(a), 24(b). The connectors 24(a), 24(b) and the holes 23 may have any other shape or configuration sufficient to allow the adhesive 32 to both adhere to the patient's skin and to prevent slippage of the connectors 24(a), 24(b) from underneath the anchor pads 22(a), 22(b). For example, a higher-strength adhesive may be provided in selected portions of the anchor pads 22(a), 22(b), such as in the region of the connectors 24(a), 24(b), or only in the regions of the holes 23.

The connectors 24(a), 24(b) need not extend underneath the anchor pads 22(a), 22(b), but may be attached to the anchor pads 22(a), 22(b) in any other configuration and by any other means consistent with their intended use. For example, the connectors 24(a), 24(b) may be attached to the upper surfaces of the anchor pads 22(a), 22(b) using hook-and-loop fasteners, or by a series of interengaging holes and protrusions. Such a configuration may allow for adjustment of the length of one or both of the connectors 24(a), 24(b) to achieve the desired longitudinal lengths of the anchors 20(a), 20(b).

One or both of the connectors 24(a), 24(b) may have a sufficient length to allow the retainers 26(a), 26(b) to clear any other medical articles or obstructing anatomy before gripping the medical line 12. In the illustrated umbilical application, for example, the connectors 24(a), 24(b) are each long enough to allow the retainers 26(a), 26(b) to grip the medical line above the umbilical stump. The connectors 24(a), 24(b) may further have a width sufficient to resist bending in the transverse direction when the anchors 20(a), 20(b) are applied (see FIG. 1) in order to maintain the desired position of the medical line 12.

The retainers 26(a), 26(b) may comprise the same material as the connectors 24(a), 24(b). The retainers 26(a), 26(b) may have a widened shape configured to allow the retainers 26(a), 26(b) to wrap at least partially around the medical line 12. The retainers 26(a), 26(b) may have a width shorter than, equal to, or longer than the outer circumference of the medical line 12, and thus may be configured to wrap only partially around, approximately once around, or more than once around the medical line 12.

To facilitate retention of the medical line 12, the undersides of the retainers 26(a), 26(b) are provided with an adhesive 34 (indicated in dashed lines). For an extremely secure retention of the medical line 12, the adhesive 34 provided on the undersides of the retainers 26(a), 26(b) may comprise a very strong adhesive, such as a VHB™ adhesive provided by 3M Corporation. To facilitate manufacturing and/or to prevent the retainers 26(a), 26(b) from sticking to the medical practitioner's hands or to other nearby medical articles, the adhesive 34 may be disposed on only a portion of the bottom surface of the retainers 26(a), 26(b), for example leaving a non-sticky border around the edges of the retainers 26(a), 26(b) when the liners 46 are removed. Alternatively or in addition, the adhesive 34 may be provided in a discontinuous pattern, such as a striped or dotted pattern, on the undersides of the retainers 26(a), 26(b).

Each retainer 26(a), 26(b) may include one or more types of adhesives. The adhesives may have different material properties, including for example adhesive strength or peal strength. Each retainer 26(a), 26(b) may include a first adhesive in a first region of the retainer and a second adhesive in a second region of the same retainer. The first and second regions may lie adjacent to each other or partially overlap.

For embodiments having overlapping adhesives, the first adhesive may cover the entire bottom surface of the retainer 26(a), 26(b) with the second adhesive covering only a portion of the bottom surface of the first adhesive. This may be advantageous for manufacturing purposes since it ensures that the entire bottom surface is covered with adhesive. Alternatively, the first adhesive may cover a first region of the retainer, such as the outer perimeter, with the second adhesive being disposed so as to be adjacent to the first region and cover the central region of the retainer. With adjacent as opposed to overlapping adhesives, the manufacturing process may require additional masking to ensure that the adhesives do not overlap.

Having multiple types of adhesives on a single retainer 26(a), 26(b) may be advantageous for handling purposes or to provide higher peal strength between the medical line 12 and the securement device 10 in a select region of the retainer 26(a), 26(b). For example, a high strength adhesive may be disposed in a center region and a lower strength adhesive disposed outside of the center region. This arrangement may be advantageous for handling purposes since only the high strength adhesive is exposed in the center region making it less likely that the healthcare provider would come in contact with the high strength adhesive.

The locations of the different adhesives may also be selected to make a selected region of the retainer 26(a), 26(b) less likely to separate from the medical line 12. The locations of the different adhesives may also be selected to further inhibit unwanted longitudinal movement of the medical line 12. For example, a higher-strength adhesive may be employed near the regions of the retainer 26(a), 26(b) that are adjacent to the connectors 24(a), 24(b) to prevent separation of the anchors 20(a), 20(b) from the medical line 12. The regions of the retainers 26(a), 26(b) which connect with the connectors 24(a), 24(b) may be exposed to higher tensile loads and accordingly may employ an adhesive with a higher peal strength. In another embodiment, regions of the retainer 26(a), 26(b) near the connectors 24(a), 24(b) and outer perimeters of the retainer 26(a), 26(b) are covered with the higher-strength adhesive 34 while the remaining regions are covered with a low-strength adhesive.

Further, the retainers 26(a), 26(b) may be provided with a tab portion without adhesive on its underside, configured to facilitate application and/or removal of the retainers 26(a), 26(b) from the medical line 12.

As mentioned above, the retainers 26(a), 26(b) may be configured to grip the medical line 12 at separate locations along the length of the medical line 12. The retainers 26(a), 26(b) may also be configured to grip the medical line from different approaches, that is, from different angles with respect to the insertion site. In such a configuration, the connector 24(b) may be longer than the connector 24(a). In alternative embodiments, however, the retainers 26(a), 26(b) may be configured to grip the medical line 12 at approximately the same location along the length of the line 12 (but from different approaches). In these and other embodiments, the retainers 26(a), 26(b) may be configured to wrap around one another or to otherwise engage each other and grip the medical line 12. Further, although illustrated with retainers 26(a), 26(b) comprising flexible portions having adhesive disposed on one side, it will be understood by one of skill in the art that the retainers 26(a), 26(b) can have any configuration suitable for limiting longitudinal, lateral, and/or transverse movement of the medical line 12. For example, the retainers may comprise clips configured to constrain longitudinal movement of the medical line 12. The clips may be configured to grip the medical line at separate locations, or may be configured to grip the medical line 12 at approximately the same location along the length of the medical line 12. In the latter embodiment, the clips may be configured to engage each other as well as the medical line 12. The retainers may further be provided with one or more additional features configured to interact with engaging structure provided on the medical line 12. For example, retainers comprising clips may include internal or external extensions having a radial component configured to abut corresponding surfaces on the medical line 12.

To use the device 10 in a medical application, a medical practitioner may first establish the appropriate position of an indwelling medical line 12 relative to a patient, according to known procedures. The practitioner may then remove the liner 42 from one of the anchor pads 22(a), 22(b), for example pad 22(a) having the shorter connector 24(a), and place the pad 22(a) on the neonate's skin to one side of the insertion site (or above or below, depending on the application). The pad 22(a) may be positioned so that the connector 24(a) extends toward the insertion site (i.e., toward the medical line 12). The medical practitioner may then remove the liner 42 from the other anchor pad 22(b) and, using the shape of the pads or other indicators such as the arrows 21 as a guide, place the second pad 22(b) in a generally opposing relationship to the first pad 22(a) with the connector 24(b) extending toward the insertion site.

Next, the practitioner may bend the connector 24(a) away from the surface of the patient's body and secure the retainer 26(a) to the medical line 12. In the umbilical application illustrated in FIGS. 1-7, for example, the previous step will include bending the connector 24(a) in an upward direction, removing the liner 46 from the retainer 26(a), and wrapping the adhesive portion 36 around the medical line 12 using the tab portion 28(a). The practitioner may then secure the retainer 26(b) to the medical line 12 in a similar fashion. In the illustrated example, the practitioner will secure the retainer 26(b) at a location further away from the patient's body than the retainer 26(a) (see FIGS. 1 and 2).

Alternatively, the practitioner may secure the retainer 24(a) to the medical line 12 prior to placing the anchor 22(b) on the patient. Additionally, the practitioner may secure one or both retainers 24(a), 24(b) to the medical line 12 prior to placing the anchors 22(a), 22(b) on the patient. In applications requiring additional stabilization of the medical line 12, of course, more than two anchors can be disposed at various locations about an insertion site so as to grip the medical line 12 from various approaches.

The various embodiments of securement devices and techniques described above thus provide a number of ways to provide safe and releasable securement for medical articles to the skin of a patient. In addition, the techniques described may be broadly applied for use with a variety of medical lines and medical procedures.

Of course, it is to be understood that not necessarily all such objectives or advantages may be achieved in accordance with any particular embodiment using the systems described herein. Thus, for example, those skilled in the art will recognize that the systems may be developed in a manner that achieves or optimizes one advantage or group of advantages as taught herein without necessarily achieving other objectives or advantages as may be taught or suggested herein.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In particular, while the present anchor has been described in the context of particularly preferred embodiments, the skilled artisan will appreciate, in view of the present disclosure, that certain advantages, features and aspects of the anchor may be realized in a variety of other applications, many of which have been noted above. For example, while particularly useful for small-scale applications, such as the illustrated medical application, the skilled artisan can readily adopt the principles and advantages described herein to a variety of other applications, including larger scale devices.

Additionally, it is contemplated that various aspects and features of the invention described can be practiced separately, combined together, or substituted for one another, and that a variety of combination and subcombinations of the features and aspects can be made and still fall within the scope of the invention. Thus, it is intended that the scope of the present invention herein disclosed should not be limited by the particular disclosed embodiments described above, but should be determined only by a fair reading of the claims that follow.

What is claimed is:

1. A securement device for securing a medical line having a longitudinal axis in a generally upright manner near an insertion site, the device comprising:
a first anchor having a first anchor pad, a first retainer, and a first flexible connector extending between the first anchor pad and the first retainer, the first retainer being configured to grip the medical line at a first location along a longitudinal axis; and
a second anchor having a second anchor pad, a second retainer, and a second flexible connector extending between the second anchor pad and the second retainer, the second retainer being configured to grip the medical line at a second location along the longitudinal axis different than the first location, the first and second flexible connectors being radially offset from each other about the longitudinal axis of the medical line at least when the medical line is secured to the patient.

2. The securement device of claim 1, wherein the second location is distal of the first location with respect to the insertion site.

3. The securement device of claim 2, wherein the second flexible connector is longer than the first flexible connector.

4. The securement device of claim 3, wherein the second flexible connector is longer than the first flexible connector by at least a longitudinal length of the first retainer.

5. The securement device of claim 1, wherein the first and second locations are approximately equidistant from the insertion site.

6. The securement device of claim 1, wherein the first connector is offset from the second connector by at least 50°.

7. The securement device of claim 6, wherein the first connector is in an opposing relationship to the second connector.

8. The securement device of claim 1, wherein the first and second retainers comprise polyester film.

9. The securement device of claim 1, wherein the first and second retainers comprise the same material as the first and second flexible connectors.

10. The securement device of claim 1, wherein the first retainer and the first connector are unitary.

11. The securement device of claim 1, wherein the first and second retainers further comprise an adhesive layer.

12. A method of securing a medical line having a longitudinal axis in a generally upright manner near an insertion site, the method comprising:
securing a first anchor to a patient, the first anchor having a first anchor pad, a first retainer, and a first flexible connector extending between the first anchor pad and the first retainer, the first retainer being configured to grip the medical line at a first location along the longitudinal axis of the medical line; and
securing a second anchor to a patient at a different angle with respect to the insertion site than the first anchor, the second anchor having a second anchor pad, a second retainer, and a second flexible connector extending between the second anchor pad and the second retainer, the second retainer being configured to grip the medical line at a second location along the longitudinal axis of the medical line.

13. The method of claim 12, wherein the second anchor is secured at least 50° from the first anchor about the insertion site.

14. The method of claim 13, wherein the second anchor is secured in an opposing relationship to the first anchor about the insertion site.

15. A securement device for securing a medical line having a longitudinal axis in a generally upright manner near an insertion site, the device comprising:
an anchor pad;
a retainer configured to adhere to the medical line along a longitudinal axis;
a flexible connector between the anchor pad and the retainer; and
a first adhesive and a second adhesive.

16. The securement device of claim 15, wherein the first adhesive has different peal strength than the second adhesive.

17. The securement device of claim 15, wherein the first adhesive and the second adhesive are disposed on the retainer.

18. The securement device of claim 15, wherein the first adhesive and the second adhesive are disposed on the anchor pad.

19. The securement device of claim 15, wherein the first adhesive at least partially overlaps the second adhesive.

20. The securement device of claim 15, wherein the first adhesive is disposed in a center region of the anchor pad and the second adhesive is disposed around a perimeter of the anchor pad.

21. The securement device of claim 15, wherein the flexible connector is configured to allow the retainer to adhere to a surface that is perpendicular to the anchor pad.

22. The securement device of claim 15, wherein the first adhesive is VHB™.

23. A securement device for securing a medical line having a longitudinal axis in a generally upright manner near an insertion site, the device comprising:
an anchor pad having an adhesive on at least a portion of a lower surface;
a retainer configured to adhere to the medical line along a longitudinal axis;
a flexible connector between the anchor pad and the retainer, the flexible connector having at least one receptacle through which at least a portion of the adhesive directly adheres the anchor pad to the patient.

24. The securement device of claim 23, wherein the receptacle is a hole.

25. The securement device of claim 23, wherein the flexible connector comprises a series of receptacles.
